(19) United States
(12) Patent Application Publication
(10) Pub. No.: US 2021/0236394 A1
(43) Pub. Date: Aug. 5, 2021

(54) METHOD OF PREPARING ENCAPSULATED LIQUID COMPOSITION

(71) Applicant: The Procter & Gamble Company, Cincinnati, OH (US)

(72) Inventors: Shufen FAN, Singapore (SG); Kenneth Shun Qiang ANG, Singapore (SG); YunQin LEE, Singapore (SG)

(21) Appl. No.: 17/239,799

(22) Filed: Apr. 26, 2021

Related U.S. Application Data
(63) Continuation of application No. PCT/US2019/057747, filed on Oct. 24, 2019.
(60) Provisional application No. 62/751,711, filed on Oct. 29, 2018.
(60) Provisional application No. 62/790,469, filed on Jan. 10, 2019.
(60) Provisional application No. 62/807,762, filed on Feb. 20, 2019.

(51) Int. Cl. A61K 8/11; A61Q 5/02; A61Q 5/12; A61K 8/73

(52) U.S. Cl. CPC A61K 8/11; A61K 8/733; A61Q 5/12; A61Q 5/02

(57) ABSTRACT

Disclosed is a method of preparing an encapsulated liquid composition comprising the steps: preparing a frozen shaped unit of a liquid composition; and forming a shell to encapsulate the frozen shaped unit. The shell is formed by, for example, an alginate gel. The method of the present invention is suitable to provide, for example, bigger capsules and/or shaped capsules even for liquid compositions with low viscosity.

### FIELD OF THE INVENTION

The present invention relates to a method of preparing an encapsulated liquid composition comprising the steps: Preparing a frozen shaped unit of a liquid composition; and forming a shell to encapsulate the frozen shaped unit. The shell is formed by, for example, an alginate gel. The method of the present invention is suitable to provide, for example, bigger capsules and/or shaped capsules even for liquid compositions with low viscosity.

### BACKGROUND OF THE INVENTION

Calcium alginate capsules have been used in a wide range of industries such as cosmetics and foods with a variety of sizes.

For example, Brian G. Zukas et. al discloses the capsules with about 10 mm diameter, in their articles titled “Improved Water Barrier Properties of Calcium Alginate Capsules Modified by Silicone Oil” (Gels 2016, 2(2), 14; doi:10.3390/gels2020014).

Another example could be U.S. Pat. No. 4,765,984, which discloses the use of the alginate/calcium gelation reaction in the formation of single unit dose dental products.

One more example could be from MAILONLINE web magazine article titled “Will this be the end of plastic bottles? Edible water ‘balloon’ that looks like a breast implant will go on sale within a year” (PUBLISHED: 16:13 BST, 11 Apr. 2017|UPDATED: 16:23 BST, 11 Apr. 2017). This article introduces seaweed spheres created using sodium alginate and calcium chloride, which contains nine ounces (250 ml) of water.

However, in such capsules, there still remains a need to provides at least one of the following:
Providing an encapsulated liquid composition in a bigger size, even for the liquid compositions with a lower viscosity;
Providing an encapsulated liquid composition in other shapes than spherical shape, even for the liquid composition with a lower viscosity;
Providing an encapsulated liquid composition with a variety of aesthetics, for example: multi-color such as phased, stripe, marble, and rainbow; and dispersing colored objects;
Providing an encapsulated liquid composition while containing immiscible, incompatible and/or reactive material;
Providing an encapsulated liquid composition for any type of liquid composition including anhydrous liquid compositions; and
Providing an encapsulated liquid composition while reducing potential interaction between the liquid composition and alginates/source of calcium cations.

None of the existing art provides all of the advantages and benefits of the present invention.

### SUMMARY OF THE INVENTION

The present invention is directed to a method of preparing an encapsulated liquid composition comprising the steps:

Preparing a frozen shaped unit of a liquid composition; and
Forming a shell to encapsulate the frozen shaped unit.

The shell is preferably formed by an alginate gel. In such case, the method of preparing the encapsulated liquid composition preferably comprising the steps:
(1) Preparing a frozen shaped unit of a liquid composition, wherein the frozen shaped unit also contains an alginate or a source of divalent and/or trivalent cation;
(2) Preparing an aqueous solution comprising an alginate or a source of divalent and/or trivalent cation which is not contained in the frozen shaped unit;
(3) Immersing the frozen shaped unit into the aqueous solution;
(4) Forming alginate gel shell by the alginate and the source of divalent and/or trivalent cation to encapsulate the frozen shaped unit.

The present invention provides at least one of the following benefits:
Providing an encapsulated liquid composition in a bigger size, even for the liquid compositions with a lower viscosity;
Providing an encapsulated liquid composition in other shapes than spherical shape, even for the liquid composition with a lower viscosity;
Providing an encapsulated liquid composition with a variety of aesthetics, for example: multi-color such as phased, stripe, marble, and rainbow; and dispersing colored objects;
Providing an encapsulated liquid composition while containing immiscible, incompatible and/or reactive material;
Providing an encapsulated liquid composition for any type of liquid composition including anhydrous liquid compositions, especially when coating alginates or source of divalent and/or trivalent cations on the frozen shaped unit; and
Providing an encapsulated liquid composition while reducing potential interaction between the liquid composition and alginates/source of divalent and/or trivalent cations, especially when coating alginates or source of divalent and/or trivalent cations on the frozen shaped unit.

None of the existing art provides all of the advantages and benefits of the present invention. These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

The term “molecular weight” or “M.Wt.” as used herein refers to the weight average molecular weight unless otherwise stated.

### Frozen Shell Encapsulation Method

The method of preparing an encapsulated liquid composition of the present invention comprising the steps:

Preparing a frozen shaped unit of a liquid composition; and
Forming a shell to encapsulate the frozen shaped unit.

The encapsulated liquid composition comprises a shaped shell and the liquid composition inside of the shell. The shape of the shell is formed according to the shape of the frozen shaped unit.

The shell can be made by any materials, preferably a material selected from the group consisting of alginate gel, carrageenan gel, chitosan gel, or mixtures thereof, more preferably alginate gel. These gels are usually formed by ionic crosslinking reaction between a polymer such as alginate, carrageenan and chitosan, and ions or source of ions.

The shell and encapsulation can be formed by any means. For alginate gel shell and encapsulation, its preparation method is explained below in detail.

“Frozen shaped unit” and “liquid composition” are also explained below in detail.

### Frozen Alginate Gel Shell Encapsulation Method

The method of preparing an encapsulated liquid composition preferably comprises the steps:
(1) Preparing a frozen shaped unit of a liquid composition, wherein the frozen shaped unit also contains an alginate or a source of divalent and/or trivalent cation;
(2) Preparing an aqueous solution comprising an alginate or a source of divalent and/or trivalent cation which is not contained in the frozen shaped unit;
(3) Immersing the frozen shaped unit into the aqueous solution;
(4) Forming alginate gel shell by the alginate and the source of divalent and/or trivalent cation to encapsulate the frozen shaped unit.

### Injection

The method of the present invention may further comprise a step of injecting a 2^{nd} composition inside the shell. The 2^{nd} composition is preferably different from the liquid composition inside of the shell, more preferably is visibly distinct from the liquid composition inside of the shell. This injection step may be preferred especially when the 2^{nd} composition is:
Not frozen at conventional conditions, for example, liquid compositions containing higher amounts of alcohols having lower freezing point than that of water, liquid compositions containing higher amounts of oily compounds having lower freezing point than that of water, and also gaseous composition such as oxygen and carbon dioxide;
Containing any temperature sensitive materials such as flavor and/or scent; and
Immiscible to the liquid composition inside of the shell;

### Capsule in Capsule

The method of the present invention can also be used for making “capsule in capsule”, by containing an encapsulated 3rd composition in the frozen shaped unit in the above step (1). This capsule in capsule may be preferred especially for:
Aesthetic improvement, for example, such that the smaller inner colored capsules are dispersed in the liquid composition in a bigger outer capsule; and/or
Stability improvement, for example, such that a smaller inner capsule can contain material which is immiscible, incompatible and/or reactive with material in the liquid composition outside of the smaller inner capsule but inside of the bigger outer capsule, including, but not limited to gaseous material.
Delayed delivery of the benefits of the 3^{rd} composition and/or the benefit of the mixture of the 3^{rd} composition and the liquid composition, after delivering the benefit of the liquid composition, for example when using shampoo as the liquid composition and conditioner as the 3^{rd} composition.
   The 3^{rd} composition can be same or different from the liquid composition.
   Alginate/Source of Divalent and/or Trivalent Cation

The present invention uses crosslinking reaction between the alginate and the source of divalent and/or trivalent cation to form alginate gel shell to encapsulate the frozen shaped unit.

In the method of the present invention, the alginate or source of divalent/trivalent cation can be either: mixed in the liquid composition; or coated on the frozen shaped unit. Such coating can be provided by, for example, spray coating or dip coating. Coating may be preferred than mixing, especially when the liquid composition is anhydrous. Coating may also be preferred than mixing, for reducing any potential interaction between the liquid composition and the alginate/source of the cations.

In the present invention, it is preferred that the frozen shaped unit contains the source of divalent and/or trivalent cation, and the aqueous solution comprises the alginate.

In the present invention, the source of divalent cation includes, for example, sources of Mn, Zn, Ni, Co, Fe, Mg, Ca, Sr, Ba, Cd, Cu, Pb divalent cations, and the source of trivalent cation include, for example, sources of Fe, Cr, Al, Ga, Sc, and La trivalent cations. The present invention can use one or more sources of divalent cations, trivalent cations, and mixtures thereof. Among them, preferred are the source of divalent cations. More preferably, the divalent cations are selected from the group consisting of Ca divalent cation, Mg divalent cation and mixtures thereof, still more preferably Ca divalent cation.

It is also preferred that the source of divalent and/or trivalent cation have at least 0.0013% solubility in water, more preferably 1% solubility in water, and still more preferably 5% solubility in water.

The sources of Ca divalent cation having at least 5% solubility in water include, for example, calcium chloride, calcium lactate, Calcium Lactate Gluconate, calcium sulfate, calcium carbonate, dicalcium phosphate and the like, preferably, calcium chloride, calcium lactate, Calcium Lactate Gluconate.

The amount of the source of divalent and/or trivalent cation can be adjusted depending on how it's used, for example, whether it is mixed with the liquid composition, whether it is coated on the frozen shaped unit, and/or whether it is contained in the aqueous solution. For example, when it is mixed with the liquid composition, it is contained in the liquid composition at a level of preferably from about 0.05% to about 5%, more preferably from about 0.1% to about 3% by weight of the liquid composition.

In the present invention, the alginates useful herein are for example, sodium alginate and potassium alginate, preferably sodium alginate. The amount of the source of alginates can be adjusted depending on how it's used, for example, whether it is mixed with the liquid composition, whether it is coated on the frozen shaped unit, and/or whether it is contained in the aqueous solution. When the alginate is contained in the aqueous solution, the alginate can be contained at a level up to about 2%, preferably from about 0.1% to about 1.5% and more preferably from about 0.3% to about 1.0% by weight of the aqueous solution.

The shell has a shell breakage force of preferably from about 0.1 Newton to about 100 Newton, more preferably from about 0.5 Newton to about 50 Newton, still more preferably from about 1 Newton to about 10 Newton.

The shell breakage force is measured by Motorized Compression Tester e.g. MTS Synergy/Bionix 100 or equivalent machines, with a compression plunger having a cylindrical terminal portion with a lager diameter than the diameter of capsule sample, at 23±3° C. (73±5.4° F.) and 40% RH.

The shell breakage force is measured by by the following method:

Place the capsule sample a test vessel; orient the capsule to ensure the largest capsule dimension lies flat on the vessel bottom surface;
Set the height of the plunger so that it rests slightly above (less than 1 mm) the capsule;
Select the Compression Rate (i.e., crosshead or ram speed) at 20 mm/min; and
Run test for each sample and record the peak force value as Shell breakage force.

### Frozen Shaped Unit

Frozen shaped unit herein means that the shaped unit can be fully frozen or partially frozen as long as it can keep its shape until the completion of the formation of shell.

Partially frozen means, frozen outside but liquid inside. For example, “80% frozen” means that the shaped unit is frozen from outside and 20% non-frozen liquid composition exists inside of the frozen shaped unit. When the shaped units are partially frozen, it is preferred to make it frozen more than 10%, more preferably more than 30%, still more preferably more than 50%, even more preferably more than 70%, furthermore preferably more than 80%.

“Frozen” herein means, being frozen around 0° C. for many aqueous compositions generally, however, also means being frozen and/or being solid at any temperature under the melting point of the liquid composition.

Frozen shaped unit can be prepared by any means, for example, pouring the liquid composition to a shaped mold to make it frozen in the mold, and/or preparing evenly spread sheet of the liquid composition having a certain viscosity and press the sheet by a shaped mold and make it frozen.

### Liquid Composition

Liquid compositions useful herein can be anhydrous liquid composition or aqueous liquid composition. The liquid composition has a viscosity of from about 1 cp to about 1,000,000 cp, more preferably from about 1 cp to about 500,000 cp, still more preferably from about 1 cp to about 200,000 cp. While the method of the present invention can be applied to liquid compositions with any viscosity, the method may be preferably used for liquid compositions with lower viscosity.

The liquid composition can be anything, for example, personal care composition, and non-personal care compositions such as fabric care, home care, air care and/or dish care compositions.

The liquid composition useful herein can be personal care composition which is selected from the group consisting of a hair care composition, a body care composition, a facial skin care composition, shave care, oral care compositions, personal health care, and mixtures thereof, and preferably is a hair care composition such as shampoo, conditioner, treatment, styling, growth and colorant

The liquid composition useful herein preferably has a pH of from about 3 to 9, more preferably from about 3.5 to about 7.5

### Aqueous Solution and Immersing

The aqueous solution useful herein can have a viscosity of, for example, from about 1 cp to about 600 cp, and preferably from about 10 cp to about 150 cp.

The aqueous solution may further contain viscosity adjuster, agent to adjust the spend of crosslinking reaction between alginates and the source of divalent and/or trivalent cations.

Before and during immersing, the aqueous solution has a temperature preferably up to about 50° C., more preferably up to about 40° C., still more preferably up to about 35° C., even more preferably up to about 30° C., and preferably from about 5° C., more preferably from about 10° C., still more preferably from about 15° C., even more preferably from about 20° C., in view of accelerating the crosslinking reaction between the cations and alginates (alginate gel shell formation) while keeping the frozen shape until the completion of the alginate gel shell formation.

Immersing can be continued preferably from about 1 sec to 24 hours, more preferably from about 1 sec to 1 hour, still more preferably from about 10 sec to 30 min, furthermore preferably from about 10 sec to 10 min, even more preferably from about 20 sec to 10 min.

During immersing, it's better to avoid the frozen shaped unit contacting other frozen shaped units by, for example, using separations between such frozen shaped units.

### Size/Shape/Aesthetics

The frozen shaped unit and the encapsulated liquid composition may have a diameter of at least about 100 nanometer in diameter and no upper limitation, preferably from about 0.5 micrometer to about 150 mm, more preferably from about 5 mm to about 150 mm, still more preferably from about 5 mm to about 100 mm, furthermore preferably from about 5 mm to about 60 mm. While the method of the present invention can be applied to make any size of capsules, the method may be preferably used to make a bigger size of capsules, such as those having a diameter of at least about 10 mm, more preferably at least about 15 mm still more preferably at least about 20 mm, even more preferably at least about 30 mm.

The frozen shaped unit and the encapsulated liquid composition can have any shape, and may have a shape other than spherical shape. While the method of the present invention can be applied to make any shape of capsules, the method may be preferably used to make capsules having other shapes than spherical shape. Such other shapes than spherical shapes include, for example, star, heart, flower, triangle, square, cube, pyramid, diamond, rectangular, donut, and oval.

Frozen shaped unit and the encapsulated liquid composition can contain one liquid composition or two or more liquid compositions. When containing two or more liquid compositions especially when such two or more liquid compositions have different colors, it may be preferred that such two or more liquid compositions have certain viscosities to not mixed in the capsule.

The liquid composition inside of the shell can have a variety of aesthetics, for example: homogeneous single color; multi-color such as phased, stripe, marble, and rainbow, wherein such multi-color can be made by two or more liquid compositions having different colors, or one liquid composition having different colors; dispersing colored objects such as flower petals and smaller inner capsules with a variety of size, shape and color; changing color depending on environmental matters (such as temperature, pH, pressure, moisture and humidity). While the method of the present invention can be applied to make capsules with any of the above aesthetics, the method may be preferably used to make capsules with the following aesthetics: multi-color such as phased, stripe, marble, and rainbow; dispersing colored objects such as flower petals for aesthetic, encapsulated liquid composition for stability by containing incompatible material inside of the encapsulated liquid composition.

The shell also can have a variety of aesthetics, for example: homogeneous single color with or without containing dyes and/or mica; multi-color such as phased, stripe, marble, and rainbow; changing color depending on environmental matters (such as temperature, pH, pressure, moisture and humidity).

### Container for the Encapsulated Liquid Composition

The encapsulated liquid composition can be contained in a container for sales to consumers. One capsule of the liquid composition can be contained in one container, mainly for single dose of the composition. Alternatively, two or more capsules can be contained in one container. Such capsule or capsules can be contained in a container with or without liquid carrier to suspend the capsule or the capsules. Such liquid carriers can be anything as long as such liquid carriers have compatibility with the shell, for example, not to dissolve or break the shell. Such liquid carriers include, for example: water and other aqueous carriers such as ethanol, anhydrous liquid carrier such as oils; and same or different liquid composition from liquid composition inside of the capsule; preferably, those with preservatives.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Liquid Composition Example 1: Aqueous Hair Shampoo Liquid Composition

Water, sodium laureth sulfate, sodium lauryl sulfate, cocamidopropyl betaine, sodium chloride, citric acid, sodium citrate, sodium xylenesulfonate, fragrance, cocamide MEA, sodium benzoate, PEG-60 almond glycerides, trisodium ethylenediamine disuccinate, tetrasodium EDTA, guar hydroxypropyltrimonium chloride, panthenol, panthenyl ethyl ether, methylchloroisothiazolinone, methylisothiazolinone.

### Liquid Composition Example 2: Aqueous Hair Conditioner Liquid Composition

Water, stearyl alcohol, behentrimonium methosulfate, bis-aminopropyl dimethicone, cetyl alcohol, fragrance, benzyl alcohol, dicetyldimonium chloride, isopropyl alcohol, disodium EDTA, propylene glycol, citric acid, histidine, butylene glycol, aloe barbadensis leaf juice, argania spinose kernel oil, alcohol denatured, methylchloroisothiazolinone, methylisothiazoline, ecklonia radiata extract.

### Liquid Composition Example 3: Aqueous Hair Treatment Liquid Composition

Water, Amodimethicone, Stearyl Alcohol, Cetyl Alcohol, Stearamidopropyl Dimethylamine, Fragrance, L-Glutamic Acid, Ethylene Diamine Tetraacetic Acid, Benzyl Alcohol, Panthenyl Ether, Panthenol DL 56 Solution, Methylchloroisothiazonlinone/Methylisothiazolinone Sodium Chloride, Carthamus Tinctorius (Safflower) Seed Oil, Cocos Nucifera (Coconut) Oil, Trimethylsiloxysilicate-MQ resin, Aloe Barbadensis Leaf Juice, Citric Acid Anhydrous

### Method Example 1: Making of Encapsulated Hair Care Composition

2 wt % of calcium chloride powder is added to a hair care composition of any of the Liquid composition example 1, 2 or 3, and stirred to mix well. Separately, sodium alginate water bath at 0.5% concentration is prepared. 7 ml of the mixture of calcium chloride and the hair care composition is filled into a shaped mold and fully frozen. The frozen shaped unit is then extracted from the mold and immersed in the sodium alginate water bath for 4 min at a room temperature to allow the formation of the alginate gel shell surrounding the shaped unit. The shaped encapsulated hair care composition is then rinsed in a water bath. The shaped encapsulated hair care composition has a shaped alginate gel shell and contains the liquid hair care composition inside of the shell.

### Method Example 2: Making of Encapsulated Hair Care Composition Using Spray Coating

7 ml of a hair care composition of any of the Liquid composition example 1, 2 or 3 is filled in a shaped mold and fully frozen. Separately, sodium alginate water bath at 0.5% concentration is prepared. 2 wt % calcium chloride in deionized water is prepared and filled into a misting spray apparatus. The frozen shaped unit is extracted from the mold and the calcium chloride solution is sprayed on the frozen shaped unit to coat the frozen shaped unit with a very thin film of calcium chloride solution. The coated frozen shaped unit is then immersed in the sodium alginate water bath for 4 mins at a room temperature to allow the formation of the alginate gel shell surrounding the shaped unit. The shaped encapsulated hair care composition is then rinsed in a water bath. The shaped encapsulated hair care composition has a shaped alginate gel shell and contains the liquid hair care composition inside of the shell.

### Method Example 3: Making of Encapsulated Hair Care Composition Using Dip Coating

7 ml of a hair care composition of any of the Liquid composition example 1, 2 or 3 is filled in a shaped mold and fully frozen. Separately, sodium alginate water bath at 0.5% concentration is prepared. 2 wt. % calcium chloride solution is also prepared. The frozen shaped unit is extracted from the mold and then dipped into the calcium chloride solution for 3 sec. The frozen shaped unit coated with calcium chloride is then immersed in the sodium alginate water bath for 4 mins at a room temperature to allow the formation of the alginate gel shell surrounding the shaped unit. The shaped encapsulated hair care composition is then rinsed in a water bath. The shaped encapsulated hair care composition has a shaped alginate gel shell and contains the liquid hair care composition inside of the shell.

### Encapsulated Liquid Composition Example 4: Capsule in Capsule

### Inner capsule

2 wt % of calcium chloride powder is added to a hair care composition of any of the Liquid composition example 1, 2 or 3, and stirred to mix well, to prepare 1^{st} hair care composition. 0.5 ml of the 1^{st} hair care composition is filled into an oval shaped mold and fully frozen. Separately, sodium alginate water bath at 0.5% concentration is prepared. The frozen shaped unit is then extracted from the mold and immersed in the sodium alginate water bath for 1-3 min at a room temperature to allow the formation of the alginate gel shell. The encapsulated hair care composition having oval shaped alginate gel shell and containing the 1^{st} liquid hair care composition inside, is then rinsed in a water bath.

### Outer Capsule

1.5 wt % of calcium chloride powder and color dye is added to a hair care composition of any of the Liquid composition example 1, 2 or 3, and stirred to mix well, to prepare 2^{nd} hair care composition. 5 ml of the 2^{nd} hair care composition is filled into a donut shaped mold with one or more pieces of the above prepared inner capsules and fully frozen. The frozen shaped unit is then extracted from the mold and immersed in the sodium alginate water bath for 5 min at a room temperature to allow the formation of the alginate gel shell. The multi-encapsulated hair care composition, having a donut shaped outer alginate gel shell capsule, and containing the 2^{nd} liquid hair composition and oval shaped inner capsules inside the donut shaped outer shell, is then rinsed in a water bath.

### Encapsulated Liquid Composition Example 5: Stripe

2 wt % of calcium chloride powder is added to a hair care composition of the Liquid composition example 2, and stirred to mix well. Blue color dye and glycerin are also added to the above composition and mixed well, to prepare 1^{st} hair care composition with a higher density. 3 ml of the 1^{st} hair care composition (having blue color and higher density) is filled into a shaped mold and frozen. Separately, 2 wt % of calcium chloride powder is added to a hair care composition of the Liquid composition example 2, and stirred to mix well, to prepare 2^{nd} hair care composition having white opaque color and lower density. 2 ml of the 2^{nd} hair care composition (having white opaque color and lighter density) is added on top of the frozen shaped 1^{st} hair care composition and place in freezer till fully frozen to prepare a frozen shaped unit. Separately, sodium alginate water bath at 0.5% concentration is prepared. The frozen shaped unit is then extracted from the mold and immersed in the sodium alginate water bath for 5 min at a room temperature to allow the formation of the alginate gel shell surrounding the shaped unit. The shaped encapsulated hair care composition is then rinsed in a water bath. The shaped encapsulated hair care composition has a shaped alginate gel shell and contains the 1^{st} and 2^{nd} liquid hair care compositions inside of the shell, and the 1^{st} and 2^{nd} liquid hair care compositions forms blue and white opaque stripes.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of preparing an encapsulated liquid composition comprising the steps:
preparing a frozen shaped unit of a liquid composition; and
forming a shell to encapsulate the frozen shaped unit.

2. The method of claim 1, wherein the shell is formed by a material selected from the group consisting of alginate gel, carrageenan gel, chitosan gel, or mixtures thereof.

3. The method of claim 1, wherein the shell is formed by alginate gel.

4. The method of claim 3 comprising the steps:
(1) Preparing a frozen shaped unit of a liquid composition, wherein the frozen shaped unit also contains an alginate or a source of divalent and/or trivalent cation;
(2) Preparing an aqueous solution comprising an alginate or a source of divalent and/or trivalent cation which is not contained in the frozen shaped unit;
(3) Immersing the frozen shaped unit into the aqueous solution;
(4) Forming alginate gel shell by the alginate and the source of divalent and/or trivalent cation to encapsulate the frozen shaped unit.

5. The method of claim 4, wherein the alginate or source of divalent and/or trivalent cation is either: mixed in the liquid composition; or coated on the frozen shaped unit,

6. The method of claim 4, wherein the source of divalent and/or trivalent cation is a source of divalent cation, wherein the divalent cation is selected from the group consisting of Ca divalent cation, Mg divalent cation, and mixtures thereof.

7. The method of claim 6, wherein the divalent cation is Ca divalent cation.

8. The method of claim 4, wherein the frozen shaped unit contains a source of divalent and/or trivalent cation, and the aqueous solution comprises alginate.

9. The method of claim 4, wherein the encapsulated liquid composition comprises a shaped shell and the liquid composition inside of the shell.

10. The method of claim 4, wherein the shape of the shell is formed according to the shape of the frozen shaped unit.

11. The method of claim 4, further comprising a step of injecting a 2^{nd} composition inside the shell.

12. The method of claim 11, wherein the 2^{nd} composition is different from the liquid composition inside of the alginate gel shell, and visibly distinct from the liquid composition inside of the shell.

13. The method of claim 11, wherein the frozen shaped unit further contains an encapsulated 3^{rd} composition.

14. The method of claim 4, wherein the liquid composition has a viscosity of from about 1 cp to about 1,000,000 cp.

15. The method of claim 4, wherein the frozen shaped unit and the encapsulated liquid composition have a diameter of at least about 20 mm.

16. The method of claim 4, wherein the frozen shaped unit and the encapsulated liquid composition have a shape other than spherical shape.
